(19) United States
(12) Patent Application Publication
(10) Pub. No.: US 2022/0265543 A1
(43) Pub. Date: Aug. 25, 2022

(54) COSMETIC COMPOSITIONS AND USES THEREOF

(71) Applicant: Alumier Europe Limited, Valletta (MT)

(72) Inventors: Karl LINTNER, Valletta (MT); Mindy S. GOLDSTEIN, Valletta (MT); Irwin PALEFSKY, Valletta (MT); Francine GERSTEIN, Valletta (MT); Nadeira RICKFORD, Valletta (MT)

(21) Appl. No.: 17/628,617

(22) PCT Filed: Jul. 24, 2020
(86) PCT No.: PCT/EP2020/070908

§ 371 Date: Jan. 20, 2022

(30) Foreign Application Priority Data
Jul. 24, 2019 (EP) 19305976.3

(51) Int. Cl. A61K 8/9789; A61Q 19/08; A61K 8/9794; A61K 8/06; A61K 8/67

(52) U.S. Cl. CPC A61K 8/9789; A61Q 19/08; A61K 8/9794; A61K 8/06; A61K 8/676; A61K 8/678

(57) ABSTRACT

The invention features cosmetic compositions comprising Swertia chirata extract and hydrolyzed Ceratonia siliqua seed extract formulated for topical administration and their related uses, e.g., for reducing skin aging or wrinkles.

### BACKGROUND OF THE INVENTION

With age, the texture and appearance of skin begins to deteriorate. The skin on the face, for example, can become rougher and leathery in texture, lose elasticity, become more fragile, become drier, develop wrinkles, and begin sagging. Aging of the skin may be accelerated by factors such as genetics, sun exposure, stress, obesity, harsh weather, and smoking. While there exist methods for addressing aging concerns of the skin, e.g., dermatologic surgery or anti-aging cosmetics, these methods may be expensive or ineffective at preventing or treating a person's aging concerns.

Thus there exists a need for improved cosmetics for addressing skin aging concerns.

### SUMMARY OF THE INVENTION

The present invention provides cosmetic compositions for use in treating or reducing signs of skin aging or wrinkling.

In a first aspect, the invention features a cosmetic composition including Swertia chirata extract and hydrolyzed Ceratonia siliqua seed extract formulated for topical administration. In some embodiments, the cosmetic composition further includes coumaroyl methoxytryptamine or Centaurea cyanus flower extract. The cosmetic composition may further include low molecular weight hyaluronic acid.

In a second aspect, the invention features a cosmetic composition including Swertia chirata extract and coumaroyl methoxytryptamine or Centaurea cyanus flower extract formulated for topical administration. In some embodiments, the cosmetic composition further includes low molecular weight hyaluronic acid.

In some embodiments of any of the preceding aspects, the cosmetic composition further includes an antioxidant (e.g., vitamin C and/or vitamin E). In further embodiments, the cosmetic composition further includes a collagen stimulator (e.g., bakuchiol, vitamin C, and/or N-prolyl palmitoyl tripeptide-56 acetate). In other embodiments, the cosmetic composition further includes a moisturizer (e.g., myristyl malate phosphonic acid, high molecular weight hyaluronic acid, and/or hydrolyzed Ceratonia siliqua seed extract).

In some embodiments of any of the preceding aspects, the cosmetic composition further includes an emollient (e.g., isononyl isononanoate, butylene glycol dicaprylate, and/or caprylic triglyceride), a thickener (e.g., polyquaternium-37), an emulsifier (e.g., polyquaternium-37), a humectant (e.g., propanediol), a diluent (e.g., water), a chelating agent (e.g., disodium ethylenediamine tetraacetic acid (EDTA)), a pH adjustor (e.g., citric acid and/or sodium hydroxide), and/or a preservative (e.g., gluconolactone sodium benzoate and/or caprylyl glycol ethyl hexylglycerin).

In a third aspect, the invention features a cosmetic composition including Swertia chirata extract, Ceratonia silique seed extract, Centaurea cyanus flower extract, N-prolyl palmitoyl tripeptide-56 acetate, bakuchiol, ethylhexyl palmitate, sorbitan oleate, sorbitan laurate, myristyl malate phosphonic acid, low molecular weight hyaluronic acid, high molecular weight hyaluronic acid, vitamin C, and vitamin E.

In a fourth aspect, the invention features a cosmetic composition including Swertia chirata extract, Ceratonia silique seed extract, hyaluronic acid, bakuchiol, N-prolyl palmitoyl tripeptide-56 acetate, vitamin C, vitamin E, propanediol, caprylyl glycol, ethylhexylglycerin, isononyl isononanoate, butylene glycol dicaprylate, caprylic triglyceride, ethylhexyl palmitate, sorbitan oleate, sorbitan laurate, myristyl malate phosphonic acid, polyquaternium-37, citric acid, sodium hydroxide, disodium EDTA, gluconolactone, and sodium benzoate.

In some embodiments of any of the preceding aspects, the cosmetic composition is formulated as a serum.

In a fifth aspect, the invention features a method of treating or reducing skin aging in a subject, the method including administering (e.g., topically) to the subject of the cosmetic composition of any one of the above cosmetic compositions. In some embodiments, wrinkle depth is reduced, skin tone is evened, skin texture is improved, and/or skin radiance is improved.

In a sixth aspect, the invention features a method of reducing wrinkles or the appearance of wrinkles in a subject, the method including administering (e.g., topically) to the subject of the cosmetic composition of any one of the above cosmetic compositions.

In some embodiments of the methods of the invention, administration of the cosmetic composition increases collagen, laminin, or elastin levels in the skin. In some embodiments, the cosmetic composition is administered topically. The cosmetic composition can be administered daily, for at least one week (e.g., for at least four weeks), or for between one day and eight weeks (e.g., for between two weeks and four weeks). In some embodiments, the cosmetic composition is administered to the face, hands, neck, or décolletage of the subject. In further embodiments, the cosmetic composition is used alone or in combination with one or more cosmetic or dermatological products. In particular embodiments, the subject is human.

In yet other aspects, the invention features (i) a cosmetic composition including Swertia chirata extract, hydrolyzed Ceratonia siliqua seed extract, coumaroyl methoxytryptamine, and Centaurea cyanus flower extract formulated for topical administration; (ii) a cosmetic composition including Swertia chirata extract and coumaroyl methoxytryptamine formulated for topical administration; (iii) a cosmetic composition including Swertia chirata extract and Centaurea cyanus flower extract formulated for topical administration; (iv) a cosmetic composition including Swertia chirata extract, coumaroyl methoxytryptamine, and Centaurea cyanus flower extract formulated for topical administration; (v) a cosmetic composition including hydrolyzed Ceratonia siliqua and Centaurea cyanus flower extract formulated for topical administration; (vi) a cosmetic composition including hydrolyzed Ceratonia siliqua, Centaurea cyanus flower extract, and coumaroyl methoxytryptamine formulated for topical administration; (vii) a cosmetic composition including hydrolyzed Ceratonia siliqua extract and coumaroyl methoxytryptamine or Centaurea cyanus flower extract formulated for topical administration; (viii) a cosmetic composition including hydrolyzed Ceratonia siliqua extract and coumaroyl methoxytryptamine formulated for topical administration; (ix) a cosmetic composition including Swertia chirata extract and coumaroyl methoxytryptamine formulated for topical administration.

In some embodiments of any of these other aspects, the cosmetic composition further includes an antioxidant (e.g., vitamin C and/or vitamin E). In further embodiments, the cosmetic composition further includes a collagen stimulator (e.g., bakuchiol, vitamin C, and/or N-prolyl palmitoyl tripeptide-56 acetate). In other embodiments, the cosmetic composition further includes a moisturizer (e.g., myristyl malate phosphonic acid, high molecular weight hyaluronic acid, and/or hydrolyzed Ceratonia siliqua seed extract).

In some embodiments of any of these other aspects, the cosmetic composition further includes an emollient (e.g., isononyl isononanoate, butylene glycol dicaprylate, and/or caprylic triglyceride), a thickener (e.g., polyquaternium-37), an emulsifier (e.g., polyquaternium-37), a humectant (e.g., propanediol), a diluent (e.g., water), a chelating agent (e.g., disodium ethylenediamine tetraacetic acid (EDTA)), a pH adjustor (e.g., citric acid and/or sodium hydroxide), and/or a preservative (e.g., gluconolactone sodium benzoate and/or caprylyl glycol ethylhexylglycerin).

In still another aspect, the invention features a method of treating or reducing skin aging in a subject, the method including administering (e.g., topically) to the subject of the cosmetic composition of any one of the above preceding other cosmetic compositions. In some embodiments, wrinkle depth is reduced, skin tone is evened, skin texture is improved, and/or skin radiance is improved.

In yet another aspect, the invention features a method of reducing wrinkles or the appearance of wrinkles in a subject, the method including administering (e.g., topically) to the subject of the cosmetic composition of any one of the above preceding other cosmetic compositions.

In some embodiments of the methods of the invention, administration of the cosmetic composition increases collagen, laminin, or elastin levels in the skin. In some embodiments, the cosmetic composition is administered topically. The cosmetic composition can be administered daily, for at least one week (e.g., for at least four weeks), or for between one day and eight weeks (e.g., for between two weeks and four weeks). In some embodiments, the cosmetic composition is administered to the face, hands, neck, or décolletage of the subject.

In further embodiments, the cosmetic composition is used alone or in combination with one or more cosmetic or dermatological products. In particular embodiments, the subject is human.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the terms “administering” and “administration” refer to the administration of a composition (e.g., a compound or a preparation that includes a compound as described herein) to a subject or system. Administration to an animal subject (e.g., to a human) may be by any appropriate route. For example, in some embodiments, administration may be topical.

In the practice of the methods of the present invention, an “effective amount” of any one of the compositions of the invention or a combination of any of the compositions of the invention is administered via any of the usual and acceptable methods known in the art, either singly or in combination.

The terms “increase,” “enhance,” or “activate” are all used herein to mean an increase by a statistically significant or visually apparent amount. In some embodiments, the terms “increase,” “enhance,” or “activate” can mean an increase of at least 10% as compared to a reference level, for example an increase by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more, or at least about a 2-fold, at least about a 3-fold, at least about a 4-fold, at least about a 5-fold, or at least about a 10-fold increase, or any increase between 2-fold and 10-fold or greater as compared to a reference level. An “increase” can also refer to a visible change of a factor in a subject that can be determined, e.g., by comparing before and after photos of the subject. The visual change may be measured by assigning a score to determine the level or severity of the factor to be measured (e.g., skin tone evenness).

The term “hyaluronic acid” as used herein includes both “low molecular weight hyaluronic acid” and “high molecular weight hyaluronic acid.” The term “low molecular weight hyaluronic acid” refers to hyaluronic acid having a molecular weight of less than or equal to 500 kDa. In some embodiments, the low molecular weight hyaluronic acid has a molecular weight of from 10-500 kDa, 10-50 kDa, 15-40 kDa, 30-100 kDa, 100-250 kDa, 250-400 kDa, or 400-500 kDa. The term “high molecular weight hyaluronic acid” refers to hyaluronic acid having a molecular weight of greater than 500 kDa. Typically, high molecular weight hyaluronic acid includes hyaluronic acid having a molecular weight of from 510-750 kDa, 750-1,000 kDa, 1,000-2,000 kDa, 2,000-5,000 kDa, 5,000-10,000 kDa, 10,000-15,000 kDa, or 15,000-20,000 kDa.

The terms “decrease,” “reduce,” or “inhibit” are all used herein to mean a decrease by a statistically significant or visually apparent amount. In some embodiments, “reduce,” “decrease,” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more, or at least about a 2-fold, at least about a 3-fold, at least about a 4-fold, at least about a 5-fold, or at least about a 10-fold decrease, or any decrease between 2-fold and 10-fold or greater as compared to a reference level. A “decrease” can also refer to a visible change of a factor in a subject that can be determined, e.g., by comparing before and after photos of the subject. The visual change may be measured by assigning a score to determine the level or severity of the factor to be measured (e.g., skin tone evenness).

The term “skin aging” refers to any non-pathological alteration or modification of the visual aspect or of the mechanical properties of the skin, in particular of the epidermis resulting from age, whether it is chronological and/or photo-induced. Thus, the signs of skin aging encompass, without limitation, a thinning of the skin, in particular of the epidermis, the occurrence of a micro-relief, the occurrence of fine lines and/or wrinkles on the skin, including at the lips and at the eyelids, wilting or collapse of the skin, loss of radiance of the skin, a dull complexion, circles at the eyes, a loss of density of the skin, a loss of firmness of the skin, a loss of tonicity of the skin, a loss of elasticity of the skin, an alteration of the smooth aspect of the skin, and/or an increase in the roughness of the skin.

As used herein, the term “subject” refers to any organism to which a composition in accordance with the invention may be administered. Typical subjects include any animal (e.g., mammals such as mice, rats, rabbits, non-human primates, and humans). Preferably, the subject is a human.

As used herein, the terms “treat,” “treated,” or “treating” mean both therapeutic or cosmetic treatment and prophylactic measures wherein the object is to slow down (lessen) an undesired physiological condition or obtain beneficial or desired results. Beneficial or desired results include, but are not limited to, alleviation of symptoms; diminishment of the extent of a condition; stabilized (i.e., not worsening) state of condition; delay in onset or slowing of condition; amelioration of the condition, whether detectable or undetectable; an amelioration of at least one measurable physical parameter, not necessarily discernible by the subject; or enhancement or improvement of condition. Treatment includes eliciting a statistically significant response without excessive levels of side effects. The compositions of the invention may also be used to “prevent” an undesired physiological condition.

### DETAILED DESCRIPTION OF THE INVENTION

The present invention provides cosmetic compositions suitable for treating or preventing signs of skin aging, for example, wrinkles.

### Formulations

The cosmetic compositions provided herein include the active ingredients Swertia chirata extract (also known as swertiamarin) and (i) hydrolyzed Ceratonia siliqua seed extract and/or (ii) Centaurea cyanus flower extract or coumaroyl methoxytryptamine.

In one example, the cosmetic composition includes Swertia chirata extract and Ceratonia siliqua seed extract. Such a composition may optionally further include Centaurea cyanus flower extract, coumaroyl methoxytryptamine, and/or low molecular weight hyaluronic acid.

In another example, the cosmetic composition includes Swertia chirata extract and Centaurea cyanus flower extract or coumaroyl methoxytryptamine. Such a composition may optionally further include low molecular weight hyaluronic acid.

In any of the cosmetic compositions described herein, the cosmetic composition may further include ingredients to aid with the effectiveness, texture, appearance, and/or stability of the composition. For example, the cosmetic composition may further include an antioxidant, a collagen stimulator, a moisturizer, an emollient, a thickener, an emulsifier, a humectant, a diluent, a chelating agent, a pH adjustor, and/or a preservative as described herein.

### Active Ingredients

Swertia chirata extract is used in the present invention as an anti-aging ingredient and may aid with blurring the appearance of wrinkles and providing a younger complexion. In the cosmetic compositions described herein, Swertia chirata extract may, for example, stimulate keratinocyte growth factor production from adipose-derived stem cells, improve keratinocyte production, and/or improve epidermis regeneration and thickness of the skin to which it is applied. It is available in formulations such as SWT-7™ H (maltodextrin and Swertia chirata extract) or SWT-7™ L (isopropyl palmitate, lecithin, water, and Swertia chirata extract).

Hydrolyzed Ceratonia siliqua seed extract is used in the present invention for moisturizing as well as repairing the skin. It is used in the present cosmetic compositions to help recovery of damaged skin, for example, by stimulating the migratory potential of cells as well as the synthesis of α-smooth muscle actin (α-SMA). Hydrolyzed Ceratonia siliqua seed extract is commercially available as GLYCO-REPAIR®BIO (water and hydrolyzed Ceratonia siliqua seed extract).

Centaurea cyanus flower extract and its isolated active component, coumaroyl methoxytryptamine, are used in the cosmetic compositions of the present invention for its skin radiance-enhancing or anti-aging properties. Centaurea cyanus flower extract and/or coumaroyl methoxytryptamine may control cell renewal, increase skin cell longevity, regulate inflammation mediators, and maintain the synthesis of collagen and elastin in the skin. It is commercially available in the formulations CLOTHOLINE®LV (Centaurea cyanus flower extract), CLOTHOLINE®BG (coumaroyl methoxytryptamine, poly(ethylene glycol) (PEG)-8 caprylic/capric glycerides, and butylene glycol), and CLOTHOLINE®MPD (coumaroyl methoxytryptamine and methyl propanediol).

### Other Ingredients

In addition to the active ingredients provided above, the cosmetic compositions of the invention may further include one or more of an antioxidant, a collagen stimulator, a moisturizer, an emollient, a thickener, an emulsifier, a humectant, a diluent, a chelating agent, a pH adjustor, and a preservative.

Antioxidants can be included in a cosmetic composition to prevent degradation of natural ingredients in the composition, as well as protect the skin to which the composition is applied. Exemplary antioxidants that can be used in cosmetic compositions include acai, algae extract, argan oil, butylated hydroxyanisole (BHA), butylated hydroxytoluene (BHT), coenzyme Q10, kukui nut oil, propyl gallate, Vitamin A (retinols) and derivatives thereof (e.g., tretinoin, retinaldehyde), Vitamin B₃ (niacinamide), Vitamin C and derivatives thereof (e.g., calcium ascorbate, ascorbic acid, ascorbyl glucosamine, ascorbyl glucoside, ascorbyl methylsilanol pectinate, ascorbyl palmitate, aminopropyl ascorbyl phosphate), Vitamin E and derivatives thereof (tocopherols (e.g., α-tocopherol)), and zinc. In some embodiments, the cosmetic composition includes the antioxidant Vitamin C and/or Vitamin E.

The cosmetic compositions described herein can also include, without limitation, any one of the following collagen stimulators: bakuchiol, Centaurea cyanus flower extract, coumaroyl methoxytryptamine, N-prolyl palmitoyl tripeptide-56 acetate, Vitamin A (retinols) and derivatives thereof (e.g., tretinoin, retinaldehyde), Vitamin B₃ (niacinamide), Vitamin C and derivatives thereof (e.g., calcium ascorbate, ascorbic acid, ascorbyl glucosamine, ascorbyl glucoside, ascorbyl methylsilanol pectinate, ascorbyl palmitate, aminopropyl ascorbyl phosphate), and Vitamin E and derivatives thereof (tocopherols (e.g., α-tocopherol)). In some embodiments, the cosmetic composition includes the collagen stimulator bakuchiol, Centaurea cyanus flower extract, coumaroyl methoxytryptamine, MATRIXYL® Morphomics (N-prolyl palmitoyl tripeptide-56 acetate, water, pentylene glycol, and caprylyl glycol), and/or Vitamin C.

Moisturizers that can be included in the composition include, without limitation, REVIDRATE®, hyaluronic acid (including low- and high-molecular weight hyaluronic acid), and GLYCO-REPAIR®BIO. REVDIRATE® is comprised of ethylhexyl palmitate, sorbitan oleate, sorbitan laurate, and myristyl malate phosphonic acid. Myristyl malate phosphonic acid is also known as 4-oxo-2-(phosphonooxy)-4-(tetradecyloxy)butanoic acid, 2-phosphate succinic acid, or 4-tetradecyl ester, and has the following structure:

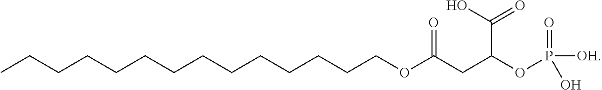 [SMILES: CCCCCCCCCCCCCCOC(=O)CC(OP(=O)(O)O)C(=O)O]

Emollients are lubricating agents that help soften the skin and seal in moisture, which creates a protective barrier on the surface. Examples of emollients that can be included in the cosmetic compositions described herein include, without limitation, benzoates, butylene glycol dicaprylate, caprylic triglyceride, cetyl alcohol, cetyl esters, cocoa butter, coconut, jojoba, sesame, almond, and other plant oils, isononyl isononanoate, lanolin, mineral oil, myristates, olive oil (oleic acid), palmitates, paraffin, petrolatum, shea butter, silicones, squalene, stearates, triethylhexanoin, and triglycerides. In some embodiments, the cosmetic composition includes the emollient isononyl isononanoate, butylene glycol dicaprylate, and/or caprylic triglyceride.

Thickeners that can be useful for the cosmetic compositions herein include agar, gellan gum, guar gum, locust bean gum, carrageenan, xanthan gum, dextran, methyl cellulose, ethyl cellulose, carboxymethyl cellulose, hydroxymethyl cellulose, hydroxyethyl cellulose, hydroxypropyl cellulose, hydroxypropyl methyl cellulose, carboxymethyl cellulose, carbomer, carboxyethyl cellulose, sodium alginate, propylene alginate glycol ester, dextran, polyvinyl alcohol, polyvinylpyrrolidone, polyvinyl methyl ether, carboxyvinyl polymer, acrylic acid-alkyl methacrylate copolymer, sodium polyacrylate, polyethylene glycol, bentonite, dextrin fatty acid ester, pectin, hydroxyethyl acrylate-sodium acryloyldimethyl taurate copolymer, dimethyl distearyl ammonium hectorite, ammonium acryloyldimethyl taurate-vinylpyrrolidone copolymer, ammonium acryloyldimethyl taurate-beheneth-25 methacrylate crosspolymer ammonium acryloyldimethyltaurate-steareth-25 methacrylate crosspolymer, polyethylene glycol distearate, polyquaternium-37, ethylene glycol triisostearate, and polyoxyethylene (20) triisostearate methyl glucoside. In some embodiments, the thickener is polyquaternium-37.

Emulsifiers in cosmetic compositions help prevent the ingredients from separating and include, for example, behentrimonium chloride, behentrimonium methosulfate, carbomer, cetaryl alcohol, cetearyl olivate, cetearyl wheat straw glucosides, emulsifying wax-NF, glyceryl stearate, laureth-4, lecithin, polyethylene glycol (PEG) stearates (e.g., PEG-2 stearate, PEG-6 stearate, PEG-8 stearate, PEG-12 stearate, PEG-20 stearate, PEG-32 stearate, PEG-40 stearate, PEG-50 stearate, PEG-100 stearate, PEG-150 stearate), polysorbates (e.g., polysorbate 80), potassium cetyl sulfate, polyquaternium-37, propylene glycol, stearic acid, stearyl alcohol, sodium stearoyl lactylate, and sorbitan olivate. In some embodiments, the emulsifier is polyquaternium-37.

Chelating agents useful for the cosmetic compositions described herein include ethylenediamine tetraacetic acid (EDTA) (e.g., Na₂-EDTA, Na₄-EDTA), gluconic acid, phytic acid, metaphosphoric acid, polyphosphoric acid, or tetrahydroxypropyl ethylenediamine. In some embodiments, the chelating agent is Na₂-EDTA.

Humectants useful for the cosmetic compositions described herein include aloe vera, alpha-hydroxy acids (e.g., glycolic acid, sorbitol, sodium hyaluronate), butylene glycol, glycerin, hyaluronic acid (including low- and high-molecular weight hyaluronic acid), propanediol, propylene glycol, lactic acid, and urea. In some embodiments, the humectant is propanediol.

pH adjustors useful for the cosmetic compositions described herein include inorganic acids (such as hydrochloric acid or sulfuric acid), organic acids (such as lactic acid, sodium lactate, citric acid, sodium citrate, succinic acid or sodium succinate), inorganic bases (such as potassium hydroxide or sodium hydroxide), and organic bases (such as triethanolamine, diisopropanolamine or triisopropanolamine). In some embodiments, the pH adjustor is citric acid and/or sodium hydroxide.

Preservatives are used in cosmetic compositions to protect a product from microbial contamination and can inhibit the growth of any given type of microorganism (e.g., bacteria, viruses, fungi, molds, or parasites) or any given microorganism (e.g., E. coli, S. aureus, or C. albicans) in the product. The preservative may also kill contaminating microorganisms and/or contribute to the composition one or more of the following attributes: stability, compatibility with other ingredients in the composition, utility at low concentrations, broad spectrum activity, and tolerability. Preferably, the preservative is hypoallergenic. Exemplary preservatives include parabens (e.g., methylparaben, propylparaben, butylparaben), phenoxyethanol, benzoic acid/sodium benzoate, sorbic acid/potassium sorbate, levulinic acid, anisic acid, isothiazolinones, gluconolactone sodium benzoate, phenylpropanol ethylhexylglycerin, caprylyl glycol, benzyl alcohol, caprylyl glycol ethylhexylglycerin. Further examples of preservatives include the preservative systems described in US 2018-0333494 A1, which is herein incorporated by reference in its entirety. In some embodiments, the cosmetic composition includes the preservative gluconolactone sodium benzoate and/or caprylyl glycol ethylhexylglycerin.

### Exemplary Formulations

In one particular exemplary formulation, the cosmetic composition described herein can include at least SWT-7™ H and GLYCO-REPAIR®BIO. In another example, the composition includes at least SWT-7™ H and CLOTHOLINE®LV. In yet another example, the composition includes at least SWT-7™ H and CLOTHOLINE®MPD. In another example, the composition includes at least SWT-7™ H, GLYCO-REPAIR®BIO, and CLOTHOLINE®LV. In yet another example, the composition includes at least SWT-7™ H, GLYCO-REPAIR®BIO, and CLOTHOLINE®MPD. In still another example, the composition includes at least SWT-7™ H, CLOTHOLINE®LV, and low molecular weight hyaluronic acid. In a further example, the composition includes SWT-7™ H, CLOTHOLINE®MPD, and low molecular weight hyaluronic acid. In any of the preceding examples, the composition may also include at least one of the following: MATRIXYL® Morphomics, bakuchiol, REVIDRATE®, low molecular weight hyaluronic acid, high molecular weight hyaluronic acid, Vitamin C (as L-ascorbic acid, BV-OSC (tetrahexyldecyl ascorbate), or Et-VC™ (3-O-ethyl ascorbic acid)), and Vitamin E.

In some embodiments of the cosmetic compositions of the invention, the cosmetic composition includes SWT-7™ H, GLYCO-REPAIR®BIO, and one or more of MATRIXYL® Morphomics, bakuchiol, REVIDRATE®, low molecular weight hyaluronic acid, high molecular weight hyaluronic acid, Vitamin C, and Vitamin E. In one example, the cosmetic composition includes SWT-7™ H, GLYCO-REPAIR®BIO, and MATRIXYL® Morphomics. In another example, the cosmetic composition includes SWT-7™ H, GLYCO-REPAIR®BIO, and bakuchiol. In another example, the cosmetic composition includes SWT-7™ H, GLYCO-REPAIR®BIO, and REVIDRATE®. In another example, the cosmetic composition includes SWT-7™ H, GLYCO-REPAIR®BIO, and low molecular weight hyaluronic acid. In another example, the cosmetic composition includes SWT-7™ H, GLYCO-REPAIR®BIO, and high molecular weight hyaluronic acid. In another example, the cosmetic composition includes SWT-7™ H, GLYCO-REPAIR®BIO, and Vitamin C. In another example, the cosmetic composition includes SWT-7™ H, GLYCO-REPAIR®BIO, and Vitamin E. In another example, the cosmetic composition includes SWT-7™ H, GLYCO-REPAIR®BIO, MATRIXYL® Morphomics, bakuchiol, REVIDRATE®, low molecular weight hyaluronic acid, high molecular weight hyaluronic acid, Vitamin C, and Vitamin E.

In other embodiments of the cosmetic compositions of the invention, the cosmetic composition includes SWT-7™ H, CLOTHOLINE®LV, and one or more of MATRIXYL® Morphomics, bakuchiol, REVIDRATE®, low molecular weight hyaluronic acid, high molecular weight hyaluronic acid, Vitamin C, and Vitamin E. In one example, the cosmetic composition includes SWT-7™ H, CLOTHOLINE®LV, and MATRIXYL® Morphomics. In another example, the cosmetic composition includes SWT-7™ H, CLOTHOLINE®LV, and bakuchiol. In another example, the cosmetic composition includes SWT-7™ H, CLOTHOLINE®LV, and REVIDRATE®. In another example, the cosmetic composition includes SWT-7™ H, CLOTHOLINE®LV, and low molecular weight hyaluronic acid. In another example, the cosmetic composition includes SWT-7™ H, CLOTHOLINE®LV, and high molecular weight hyaluronic acid. In another example, the cosmetic composition includes SWT-7™ H, CLOTHOLINE®LV, and Vitamin C. In another example, the cosmetic composition includes SWT-7™ H, CLOTHOLINE®LV, and Vitamin E. In another example, the cosmetic composition includes SWT-7™ H, CLOTHOLINE®LV, MATRIXYL® Morphomics, bakuchiol, REVIDRATE®, low molecular weight hyaluronic acid, high molecular weight hyaluronic acid, Vitamin C, and Vitamin E.

Further embodiments of the cosmetic compositions of the invention include compositions having SWT-7™ H, CLOTHOLINE®MPD, and one or more of MATRIXYL® Morphomics, bakuchiol, REVIDRATE®, low molecular weight hyaluronic acid, high molecular weight hyaluronic acid, Vitamin C, and Vitamin E. In one example, the cosmetic composition includes SWT-7™ H, CLOTHOLINE®MPD, and MATRIXYL® Morphomics. In another example, the cosmetic composition includes SWT-7™ H, CLOTHOLINE®MPD, and bakuchiol. In another example, the cosmetic composition includes SWT-7™ H, CLOTHOLINE®MPD, and REVIDRATE®. In another example, the cosmetic composition includes SWT-7™ H, CLOTHOLINE®MPD, and low molecular weight hyaluronic acid. In another example, the cosmetic composition includes SWT-7™ H, CLOTHOLINE®MPD, and high molecular weight hyaluronic acid. In another example, the cosmetic composition includes SWT-7™ H, CLOTHOLINE®MPD, and Vitamin C. In another example, the cosmetic composition includes SWT-7™ H, CLOTHOLINE®MPD, and Vitamin E. In another example, the cosmetic composition includes SWT-7™ H, CLOTHOLINE®MPD, MATRIXYL® Morphomics, bakuchiol, REVIDRATE®, low molecular weight hyaluronic acid, high molecular weight hyaluronic acid, Vitamin C, and Vitamin E.

In still further embodiments of the cosmetic compositions of the invention, the cosmetic composition includes SWT-7™ H, GLYCO-REPAIR®OBIO, CLOTHOLINE®LV, and one or more of MATRIXYL® Morphomics, bakuchiol, REVIDRATE®, low molecular weight hyaluronic acid, high molecular weight hyaluronic acid, Vitamin C, and Vitamin E. In one example, the cosmetic composition includes SWT-7™ H, GLYCO-REPAIR®BIO, CLOTHOLINE®LV, and MATRIXYL® Morphomics. In another example, the cosmetic composition includes SWT-7™ H, GLYCO-REPAIR®BIO, CLOTHOLINE®LV, and bakuchiol. In another example, the cosmetic composition includes SWT-7™ H, GLYCO-REPAIR®BIO, CLOTHOLINE®LV, and REVIDRATE®. In another example, the cosmetic composition includes SWT-7™ H, GLYCO-REPAIR®BIO, CLOTHOLINE®LV, and low molecular weight hyaluronic acid. In another example, the cosmetic composition includes SWT-7™ H, GLYCO-REPAIR®BIO, CLOTHOLINE®LV, and high molecular weight hyaluronic acid. In another example, the cosmetic composition includes SWT-7™ H, GLYCO-REPAIR®BIO, CLOTHOLINE®LV, and Vitamin C. In another example, the cosmetic composition includes SWT-7™ H, GLYCO-REPAIR®BIO, CLOTHOLINE®LV, and Vitamin E. In another example, the cosmetic composition includes SW-7™ H, GLYCO-REPAIR®BIO, CLOTHOLINE®LV, MATRIXYL® Morphomics, bakuchiol, REVIDRATE®, low molecular weight hyaluronic acid, high molecular weight hyaluronic acid, Vitamin C, and Vitamin E.

In still further embodiments of the cosmetic compositions of the invention, the cosmetic composition includes SWT-7™ H, GLYCO-REPAIR®BIO, CLOTHOLINE®MPD, and one or more of MATRIXYL® Morphomics, bakuchiol, REVIDRATE®, low molecular weight hyaluronic acid, high molecular weight hyaluronic acid, Vitamin C, and Vitamin E. In one example, the cosmetic composition includes SWT-7™ H, GLYCO-REPAIR®BIO, CLOTHOLINE®MPD, and MATRIXYL® Morphomics. In another example, the cosmetic composition includes SWT-7™ H, GLYCO-REPAIR®BIO, CLOTHOLINE®MPD, and bakuchiol. In another example, the cosmetic composition includes SWT-7™ H, GLYCO-REPAIR®BIO, CLOTHOLINE®MPD, and REVIDRATE®. In another example, the cosmetic composition includes SWT-7™ H, GLYCO-REPAIR®BIO, CLOTHOLINE®MPD, and low molecular weight hyaluronic acid. In another example, the cosmetic composition includes SWT-7™ H, GLYCO-REPAIR®BIO, CLOTHOLINE®MPD, and high molecular weight hyaluronic acid. In another example, the cosmetic composition includes SWT-7™ H, GLYCO-REPAIR®BIO, CLOTHOLINE®MPD, and Vitamin C. In another example, the cosmetic composition includes SWT-7™ H, GLYCO-REPAIR®BIO, CLOTHOLINE®MPD, and Vitamin E. In another example, the cosmetic composition includes SWT-7™ H, GLYCO-REPAIR®BIO, CLOTHOLINE®MPD, MATRIXYL® Morphomics, bakuchiol, REVIDRATE®, low molecular weight hyaluronic acid, high molecular weight hyaluronic acid, Vitamin C, and Vitamin E.

In other embodiments of the cosmetic compositions of the invention, the cosmetic composition includes SWT-7™ H, CLOTHOLINE®LV, low molecular weight hyaluronic acid, and one or more of MATRIXYL® Morphomics, bakuchiol, REVIDRATE®, high molecular weight hyaluronic acid, Vitamin C, and Vitamin E. In one example, the cosmetic composition includes SWT-7™ H, CLOTHOLINE®LV, low molecular weight hyaluronic acid, and MATRIXYL® Morphomics. In another example, the cosmetic composition includes SWT-7™ H, CLOTHOLINE®LV, low molecular weight hyaluronic acid, and bakuchiol. In another example, the cosmetic composition includes SWT-7™ H, CLOTHOLINE®LV, low molecular weight hyaluronic acid, and REVIDRATE®. In another example, the cosmetic composition includes SWT-7™ H, CLOTHOLINE®LV, low molecular weight hyaluronic acid, and high molecular weight hyaluronic acid. In another example, the cosmetic composition includes SWT-7™ H,

CLOTHOLINE®LV, low molecular weight hyaluronic acid, and Vitamin C. In another example, the cosmetic composition includes SWT-7™ H, CLOTHOLINE®LV, low molecular weight hyaluronic acid, and Vitamin E. In another example, the cosmetic composition includes SWT-7™ H, CLOTHOLINE®LV, low molecular weight hyaluronic acid, MATRIXYL® Morphomics, bakuchiol, REVIDRATE®, high molecular weight hyaluronic acid, Vitamin C, and Vitamin E.

In yet other embodiments of the cosmetic compositions of the invention, the cosmetic composition includes SWT-7™ H, CLOTHOLINE®MPD, low molecular weight hyaluronic acid, and one or more of MATRIXYL® Morphomics, bakuchiol, REVIDRATE®, high molecular weight hyaluronic acid, Vitamin C, and Vitamin E. In one example, the cosmetic composition includes SWT-7™ H, CLOTHOLINE®MPD, low molecular weight hyaluronic acid, and MATRIXYL® Morphomics. In another example, the cosmetic composition includes SWT-7™ H, CLOTHOLINE®MPD, low molecular weight hyaluronic acid, and bakuchiol. In another example, the cosmetic composition includes SWT-7™ H, CLOTHOLINE®MPD, low molecular weight hyaluronic acid, and REVIDRATE®. In another example, the cosmetic composition includes SWT-7™ H, CLOTHOLINE®MPD, low molecular weight hyaluronic acid, and high molecular weight hyaluronic acid. In another example, the cosmetic composition includes SWT-7™ H, CLOTHOLINE®MPD, low molecular weight hyaluronic acid, and Vitamin C. In another example, the cosmetic composition includes SWT-7™ H, CLOTHOLINE®MPD, low molecular weight hyaluronic acid, and Vitamin E. In another example, the cosmetic composition includes SWT-7™ H, CLOTHOLINE®MPD, low molecular weight hyaluronic acid, MATRIXYL® Morphomics, bakuchiol, REVIDRATE®, high molecular weight hyaluronic acid, Vitamin C, and Vitamin E.

In any of the preceding examples, the cosmetic composition may further include any one of the antioxidants, collagen stimulators, moisturizers, emollients, thickeners, emulsifiers, humectants, diluents, chelating agents, pH adjustors, and/or preservatives provided herein.

### Compositions

The cosmetic compositions described herein may be formulated as a lotion, a milk, a cream, an ointment, a gel, a foam, a solution, or a pomade. Furthermore, the cosmetic compositions described herein may be of any type of makeup or body product, including, e.g., a lotion, a milk, a serum, an aqueous or oily gel, an emulsion, a cream, a cream-gel, a body care water, a pomade, a balm, a foundation, a spray, an eye shadow, a stick, a lipstick, a gloss, a foam, a deodorizer, a nutritive face mask, a shower gel, and exfoliating or scrubbing product.

In certain aspects of the invention, the cosmetic composition is a serum.

### Methods of Use

The cosmetic compositions are preferably intended to be administered topically to the skin of a subject. The composition may be administered once a day, twice a day, daily or nightly, every other day, every two days, every three days, every four days, every five days, every six days, weekly, biweekly, or monthly. The composition may be administered over the course of one week, two weeks, four weeks, six weeks, eight weeks, 4 months, 6 months, 9 months, one year, or two years, or longer. Preferred areas for application include the face (e.g., cheeks, forehead, nose, chin, under the eyes), the hands, the neck, and the décolletage.

Uses for the compositions disclosed herein include treating or reducing skin aging (i.e., for anti-aging benefits) or reducing the appearance of wrinkles. The anti-aging properties of the composition can be measured by, for example, comparing before and after photos, histological evidence of positive changes (e.g., increased laminin, elastin, and/or collagen types 1, 3, and 12, reduction in elastosis, increase in epidermal or dermal thickness), wrinkle depth measurements (e.g., decrease in wrinkle depth), improved skin tone evenness, improvement in skin texture, and/or improvement in skin radiance.

In addition, the cosmetic compositions described herein may be used alone for the methods described herein, or they may be used in combination with other cosmetic or dermatological products.

### EXAMPLES

The following are examples of the methods and compositions of the invention. It is understood that various other embodiments may be practiced, given the description provided herein.

### Example 1. Anti-Aging Serum

The compositions of exemplary anti-aging serums are provided in Table 1 below.

TABLE 1
Anti-Aging Serums

| | |
|---|---|
| Ingredient | INCI Designation |

Formulation 1

| | |
|---|---|
| SWT-7 ™ H | Maltodextrin, Swertia chirata extract |
| GLYCO-REPAIR ®BIO | Water, hydrolyzed Ceratonia silique seed extract |
| CLOTHOLINE ®LV | Centaurea cyanus flower extract |
| MATRIXYL ® MORPHOMICS | Water, pentylene glycol, caprylyl glycol, N-prolyl palmitoyl |
| | tripeptide-56 acetate |
| SYTENOL ® A | Bakuchiol |
| REVIDRATE ® | Ethylhexyl palmitate, sorbitan oleate, sorbitan laurate, |
| | myristyl malate phosphonic acid |
| LOW MW HYALURONIC ACID | Low molecular weight hyaluronic acid |
| HIGH MW HYALURONIC ACID | High molecular weight hyaluronic acid |
| VITAMIN C | Vitamin C |
| VITAMIN E | Vitamin E |

Formulation 2

| | |
|---|---|
| Na₂ EDTA | Disodium EDTA |
| GEOGARD ULTRA ® | Gluconolactone, sodium benzoate |
| ZEMEA ® PROPANEDIOL | Propanediol |
| SENSIVA ® SC-10 | Caprylyl glycol, ethylhexylglycerin |
| DUB ININ (LANOL ™ 99) | Isononyl isononanoate |
| DERMOFEEL ® BGC | Butylene glycol dicaprylate |
| LIPONATE ® GC | Caprylic triglyceride |
| REVIDRATE ® | Ethylhexyl palmitate, sorbitan oleate, sorbitan laurate, |
| | myristyl malate phosphonic acid |
| VITAMIN E ACETATE CARE | Tocopheryl acetate |
| SYTENOL ® A | Bakuchiol |
| COSMEDIA ® ULTRAGEL 300 | Polyquaternium-37 |
| SWT-7 ™ H | Maltodextrin, Swertia chirata extract |
| GLYCO-REPAIR ®BIO | Water, hydrolyzed Ceratonia silique seed extract |
| MATRIXYL ® MORPHOMICS | Water, pentylene glycol, caprylyl glycol, N-prolyl palmitoyl |
| | tripeptide-56 acetate |
| K3 VITA-C | Aminopropyl ascorbyl phosphate |
| HYALURONIC ACID POWDER- | Sodium hyaluronate |
| BT | |
| PRIMALHYAL ™ 3 K | Hydrolyzed hyaluronic acid |
| CITRIC ACID 20% SOLUTION | Water, citric acid |
| SODIUMHYDROXIDE | Sodium hydroxide, water |
| PELLETS 20% SOLUTION | |

A subject applies one of the two serums described above to the skin, for example, on the face at least once a day for four weeks. As a result of application, the subject experiences reduced appearance of wrinkles on the face and an improved overall evenness of the skin tone. The subject further notices improvement in skin texture and improvement in skin radiance.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A cosmetic composition comprising Swertia chirata extract and hydrolyzed Ceratonia siliqua seed extract formulated for topical administration.

2. A cosmetic composition comprising Swertia chirata extract and coumaroyl methoxytryptamine or Centaurea cyanus flower extract formulated for topical administration.

3. The cosmetic composition of claim 1, further comprising coumaroyl methoxytryptamine or Centaurea cyanus flower extract.

4. The cosmetic composition of claims 1-3, further comprising low molecular weight hyaluronic acid.

5. The cosmetic composition of any one of claims 1-4, further comprising an antioxidant.

6. The cosmetic composition of claim 5, wherein the antioxidant comprises vitamin C and/or vitamin E.

7. The cosmetic composition of any one of claims 1-6, further comprising a collagen stimulator.

8. The cosmetic composition of claim 7, wherein the collagen stimulator comprises bakuchiol, vitamin C, and/or N-prolyl palmitoyl tripeptide-56 acetate.

9. The cosmetic composition of any one of claims 1-8, further comprising a moisturizer.

10. The cosmetic composition of claim 9, wherein the moisturizer comprises myristyl malate phosphonic acid, high molecular weight hyaluronic acid, and/or hydrolyzed Ceratonia siliqua seed extract.

11. The cosmetic composition of any one of claims 1-10, further comprising an emollient, a thickener, an emulsifier, a humectant, a diluent, a chelating agent, a pH adjustor, and/or a preservative.

12. The cosmetic composition of claim 11, wherein the emollient comprises isononyl isononanoate, butylene glycol dicaprylate, and/or caprylic triglyceride.

13. The cosmetic composition of claim 11, wherein the thickener comprises polyquaternium-37.

14. The cosmetic composition of claim 11, wherein the emulsifier comprises polyquaternium-37.

15. The cosmetic composition of claim 11, wherein the humectant comprises propanediol.

16. The cosmetic composition of claim 11, wherein the diluent comprises water.

17. The cosmetic composition of claim 11, wherein the chelating agent comprises disodium ethylenediamine tetraacetic acid (EDTA).

18. The cosmetic composition of claim 11, wherein the pH adjustor comprises citric acid and/or sodium hydroxide.

19. The cosmetic composition of claim 11, wherein the preservative comprises gluconolactone sodium benzoate and/or caprylyl glycol ethylhexylglycerin.

20. A cosmetic composition comprising Swertia chirata extract, Ceratonia silique seed extract, Centaurea cyanus flower extract, N-prolyl palmitoyl tripeptide-56 acetate, bakuchiol, ethylhexyl palmitate, sorbitan oleate, sorbitan laurate, myristyl malate phosphonic acid, low molecular weight hyaluronic acid, high molecular weight hyaluronic acid, vitamin C, and vitamin E.

21. A cosmetic composition comprising Swertia chirata extract, Ceratonia silique seed extract, hyaluronic acid, bakuchiol, N-prolyl palmitoyl tripeptide-56 acetate, vitamin C, vitamin E, propanediol, caprylyl glycol, ethylhexylglycerin, isononyl isononanoate, butylene glycol dicaprylate, caprylic triglyceride, ethylhexyl palmitate, sorbitan oleate, sorbitan laurate, myristyl malate phosphonic acid, polyquaternium-37, citric acid, sodium hydroxide, disodium EDTA, gluconolactone, and sodium benzoate.

22. The cosmetic composition of any one of claims 1-21, wherein the cosmetic composition is formulated as a serum, a lotion, a gel, a cream, an oil, a stick, a foam, a solution, an ointment, or a pomade.

23. A method of treating or reducing skin aging in a subject, the method comprising administering to the subject of the cosmetic composition of any one of claims 1-22.

24. The method of claim 23, wherein wrinkle depth is reduced, skin tone is evened, skin texture is improved, and/or skin radiance is improved.

25. A method of reducing the appearance of wrinkles in a subject, the method comprising administering to the subject of the cosmetic composition of any one of claims 1-22.

26. The method of any one of claims 23-25, wherein administration of the cosmetic composition increases collagen, laminin, or elastin levels in the skin.

27. The method of any one of claims 23-26, wherein the cosmetic composition is administered topically.

28. The method of any one of claims 23-27, wherein the cosmetic composition is administered daily.

29. The method of any one of claims 23-28, wherein the cosmetic composition is administered for at least one week.

30. The method of claim 29, wherein the cosmetic composition is administered for at least four weeks.

31. The method of any one of claims 23-28, wherein the cosmetic composition is administered for between one day and eight weeks.

32. The method of claim 31, wherein the cosmetic composition is administered for between two weeks and four weeks.

33. The method of any one of claims 23-32, wherein the cosmetic composition is administered to the face, hands, neck, or décolletage of the subject.

34. The method of any one of claims 23-33, wherein the cosmetic composition is used alone or in combination with one or more cosmetic or dermatological products.

35. The method of any one of claims 23-34, wherein the subject is human.

36. A cosmetic composition comprising Swertia chirata extract, hydrolyzed Ceratonia siliqua seed extract, coumaroyl methoxytryptamine, and Centaurea cyanus flower extract formulated for topical administration.

37. A cosmetic composition comprising Swertia chirata extract and coumaroyl methoxytryptamine formulated for topical administration.

38. A cosmetic composition comprising Swertia chirata extract and Centaurea cyanus flower extract formulated for topical administration.

39. A cosmetic composition comprising Swertia chirata extract, coumaroyl methoxytryptamine, and Centaurea cyanus flower extract formulated for topical administration.

40. A cosmetic composition comprising hydrolyzed Ceratonia siliqua and Centaurea cyanus flower extract formulated for topical administration.

41. A cosmetic composition comprising hydrolyzed Ceratonia siliqua, Centaurea cyanus flower extract, and coumaroyl methoxytryptamine formulated for topical administration.

42. A cosmetic composition comprising hydrolyzed Ceratonia siliqua extract and coumaroyl methoxytryptamine or Centaurea cyanus flower extract formulated for topical administration.

43. A cosmetic composition comprising hydrolyzed Ceratonia siliqua extract and coumaroyl methoxytryptamine formulated for topical administration.

44. A cosmetic composition comprising Swertia chirata extract and coumaroyl methoxytryptamine formulated for topical administration.

45. The cosmetic composition of claim 36-44, further comprising low molecular weight hyaluronic acid.

46. The cosmetic composition of any one of claims 36-45, further comprising an antioxidant.

47. The cosmetic composition of claim 46, wherein the antioxidant comprises vitamin C and/or vitamin E.

48. The cosmetic composition of any one of claims 36-47, further comprising a collagen stimulator.

49. The cosmetic composition of claim 48, wherein the collagen stimulator comprises bakuchiol, vitamin C, and/or N-prolyl palmitoyl tripeptide-56 acetate.

50. The cosmetic composition of any one of claims 36-49, further comprising a moisturizer.

51. The cosmetic composition of claim 50, wherein the moisturizer comprises myristyl malate phosphonic acid, high molecular weight hyaluronic acid, and/or hydrolyzed Ceratonia siliqua seed extract.

52. The cosmetic composition of any one of claims 36-51, further comprising an emollient, a thickener, an emulsifier, a humectant, a diluent, a chelating agent, a pH adjustor, and/or a preservative.

53. The cosmetic composition of claim 52, wherein the emollient comprises isononyl isononanoate, butylene glycol dicaprylate, and/or caprylic triglyceride.

54. The cosmetic composition of claim 52, wherein the thickener comprises polyquaternium-37.

55. The cosmetic composition of claim 52, wherein the emulsifier comprises polyquaternium-37.

56. The cosmetic composition of claim 52, wherein the humectant comprises propanediol.

57. The cosmetic composition of claim 52, wherein the diluent comprises water.

58. The cosmetic composition of claim 52, wherein the chelating agent comprises disodium ethylenediamine tetraacetic acid (EDTA).

59. The cosmetic composition of claim 52, wherein the pH adjustor comprises citric acid and/or sodium hydroxide.

60. The cosmetic composition of claim 52, wherein the preservative comprises gluconolactone sodium benzoate and/or caprylyl glycol ethylhexylglycerin.

61. The cosmetic composition of any one of claims 36-60, wherein the cosmetic composition is formulated as a serum, a lotion, a gel, a cream, an oil, a stick, a foam, a solution, an ointment, or a pomade.

62. A method of treating or reducing skin aging in a subject, the method comprising administering to the subject of the cosmetic composition of any one of claims 36-61.

63. The method of claim 62, wherein wrinkle depth is reduced, skin tone is evened, skin texture is improved, and/or skin radiance is improved.

64. A method of reducing the appearance of wrinkles in a subject, the method comprising administering to the subject of the cosmetic composition of any one of claims 36-61.

65. The method of any one of claims 62-64, wherein administration of the cosmetic composition increases collagen, laminin, or elastin levels in the skin.

66. The method of any one of claims 62-65, wherein the cosmetic composition is administered topically.

67. The method of any one of claims 62-66, wherein the cosmetic composition is administered daily.

68. The method of any one of claims 62-67, wherein the cosmetic composition is administered for at least one week.

69. The method of claim 68, wherein the cosmetic composition is administered for at least four weeks.

70. The method of any one of claims 62-69, wherein the cosmetic composition is administered for between one day and eight weeks.

71. The method of claim 70, wherein the cosmetic composition is administered for between two weeks and four weeks.

72. The method of any one of claims 62-71, wherein the cosmetic composition is administered to the face, hands, neck, or décolletage of the subject.

73. The method of any one of claims 62-72, wherein the cosmetic composition is used alone or in combination with one or more cosmetic or dermatological products.

74. The method of any one of claims 62-73, wherein the subject is human.
